(19) United States
(12) Patent Application Publication
(10) Pub. No.: US 2008/0274094 A1
(43) Pub. Date: Nov. 6, 2008

(54) Molecular Complex Comprising Arbutine, Ascorbic Acid, Oleuropeina or Its Derivatives Thereof and Related Uses In Medical Field

(75) Inventor: Mateo Tutino, Palermo (IT)

Correspondence Address: CASELLA & HESPOS, 274 MADISON AVENUE, NEW YORK, NY, 10016, US

(73) Assignee: MED CARE S.R.L., Palermo (IT)

(21) Appl. No.: 11/922,866

(22) PCT Filed: Jun. 19, 2006
(86) PCT No.: PCT/IT2006/000464

§ 371 Date: Dec. 26, 2007

(30) Foreign Application Priority Data
Aug. 24, 2005 (IT) RM2005A000330

(51) Int. Cl. A61K 38/44; C07D 407/12; A61K 31/351; A61K 31/403; A61K 31/07; A61K 8/49; A61K 31/355; A61K 36/886; A61K 31/7076

(52) U.S. Cl. 424/94.4; 549/415; 514/460; 514/419; 514/168; 514/458; 424/744; 514/47; 424/62

(57) ABSTRACT

The present invention relates to a molecular complex comprising arbutine, ascorbic acid, oleuropeina or its derivatives and related uses in medical field, for example for the treatment of the skin, mucosa and serosa, for the treatment of skin aging, cutaneous hyperpigmentation and skin diseases.

The present invention relates to a molecular complex comprising arbutine, ascorbic acid, oleuropeina or its derivatives and related uses in medical field. More particularly, the invention concerns compositions comprising the above mentioned molecular complex for the treatment of the skin, mucosa and serosa, for example for the treatment of skin aging, cutaneous hyperpigmentation and skin diseases.

Nowadays, alpha arbutine, a natural hydroquinone derivative, is widely used in cosmetic field as a bleaching agent since it is a tyrosinase inhibitor. The related literature and patents JP 2002265316, JP 2002145759, JP 2003277261, JP 2003277261, JP 2004115392 disclosed the use of such a compound at a concentration ranging from 0.05 to 10%. However, it is known that arbutine may induce paradoxical hyperpigmentation. To solve this problem different solutions consisting in the addition of hydroquinone, azelaic and cogic acids to the compositions containing arbutine together with other substances inhibiting thyroxine formation were suggested.

The use of arbutine in cosmetic field is recommended in variable concentrations between 0.5 to 1% up to 2%. These concentrations are standardized as during the prolonged use of arbutine, especially at dosages higher than 1-2%, paradoxical hyperpigmentation reactions may be elicited. These hyperpigmentation reactions are clearly demonstrable and are not determined by the increase of tirosinase enzyme since, also if the enzyme is inhibited, no increase of gene 1 inverse polimerase transcription related to thyroxine production is produced, but at long term (Pigment Cell Res. 1998 February; 11(1): 12-7). Arbutin increases the pigmentation of cultured human melanocytes through mechanism other than induction of tyrosinase activity). Paradoxical hyperpigmentation following use of high and chronic arbutine dosages is caused by a pre-transcriptional localized reason. Inverse transcriptase shows that there is an enhanced endothelin-1 expression and endothelin b receptors. In fact endothelins are accompanied and enhanced by an increased tirosinase mRNA expression. Furthermore, all these factors are connected to the TNFα, “tumor necrosis factor alpha” expression, that plays a crucial role in the formation of post-inflammatory hyperpigmentation, in the formation of lentigo solaris, psoriasis, tissue healing and in the acute and chronic inflammatory processes (J. Invest Dermatol. April; 116 (4):571-7. The role of epidermal endothelin cascade in the hyperpigmentation mechanism of lentigo senilis, Kadono S et Al.). Therefore, endothelin B receptor increase, plays a key role in the paradoxical hyperpigmentation or lentigo solaris as well as post-inflammatory hyperpigmentation. Furthermore, following exposure to the sun an high expression of TNF-Alpha expression inducile by ET1 cytokine, these latter being strictly related to the increased expression of TNF alpha that exerts a central role in melanogenesis thus exceeding thyroxine action.

In the light of the above it is well evident the needing of having new formulations able to overcome the drawbacks of the already known compositions comprising arbutine.

The author of the present invention has now prepared a molecular complex comprising arbutine, ascorbic acid and oleuropeina or its derivatives and, optionally, ionene polymers. This complex represents a new stable molecule, which allows to obtain the preparation of any composition, comprising an higher concentration of arbutine than known compositions without displaying paradoxical hyperpigmentation phenomena; therefore this complex represents a new molecule per se.

Particularly, by mixing alpha arbutine, ascorbic acid and oleuropeina and, optionally, ionene polymers in the same ratio in appropriate reaction conditions a tri o tetramolecular complex between these compounds was developed, wherein the hydroxyl of the phenyl group of oleuropeina reacts with the hydroxyl of ascorbic acid confers the resistance to oxidation and stability; the para hydroxyl of the phenyl group of arbutine interacts with the free hydroxyl of ascorbic acid thus forming a stable binding wherein oxygen acquires a negative charge. Therefore, trimolecular complex has the following formula (I):

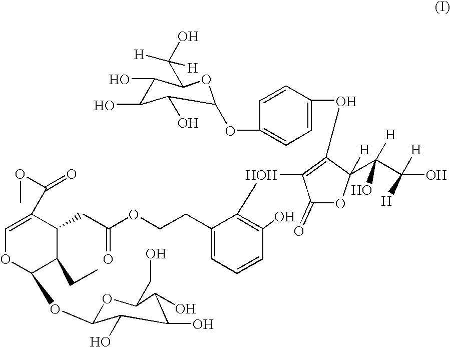

The aforementioned interactions between compounds confer more stability and effectiveness to ascorbic acid action. Furthermore, by the addition of ionene polymers, negative charge on the oxygen atom of ascorbic acid will interact with amine nitrogen of ionene polymer bearing a positive charge, thus forming a tetramolecular complex having the following formula (II):

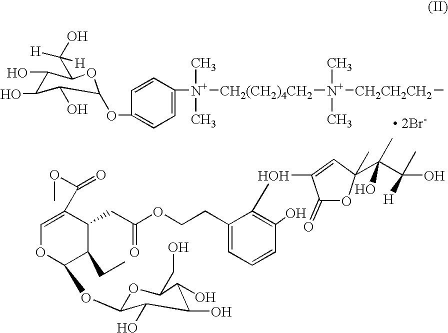

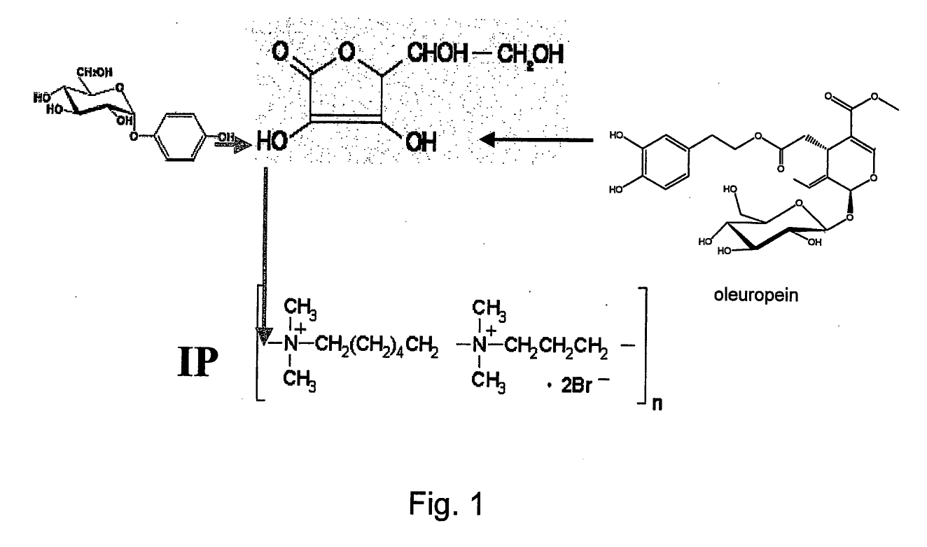

The interactions between the aforementioned compounds are depicted in FIG. 1.

In the preparations according to the invention, Alpha arbutine, Ascorbic Acid (LAA) and olea europea containing oleuropeina and, optionally, ionene polymer, concentrations should be in the same rate as the complex would form a unique molecule which acts not only by inhibiting tyrosinase enzymatic activity but at the same time shows an upstream action, by inhibiting all cascade mechanisms inducing ET1 release and other signals stimulating TNFα formation, the latter being one of the agents directly involved in paradoxical hyperpigmentation.

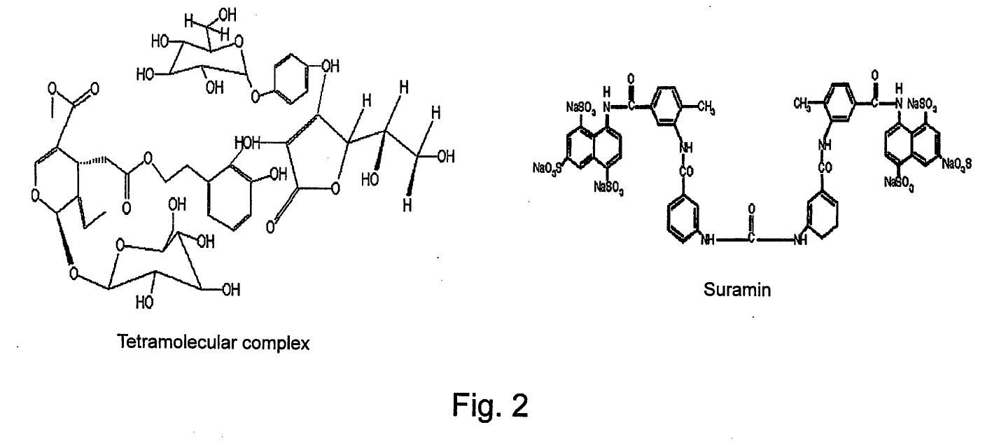

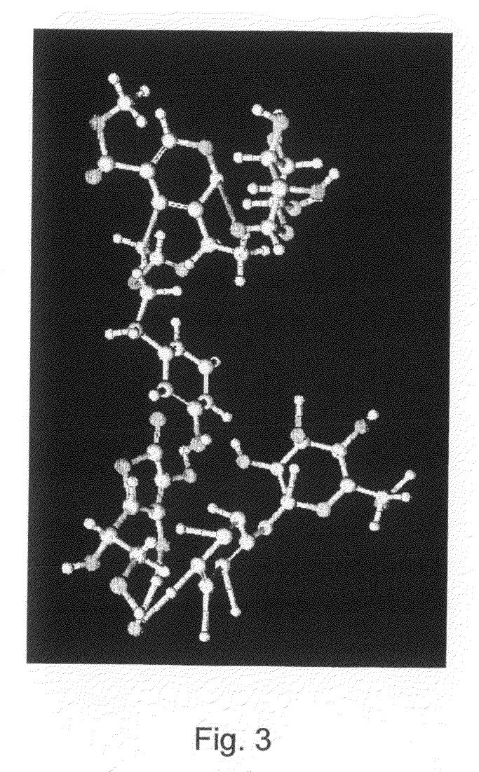

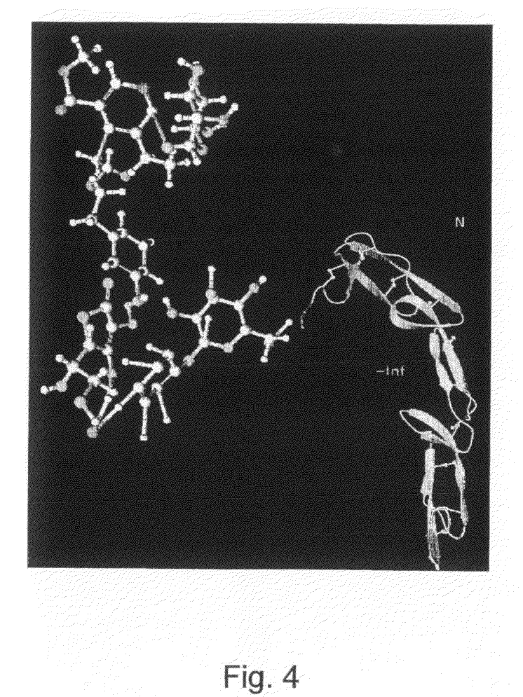

Oleuropeina, tyrosol and hydroxytyrosol, the latter two also founded in olea europea extract, induce a direct action on cytokines IL1α and TNFα involved in paradoxical hyperpigmentation, and mediate those intracellular signals elicited by ET inside melanocytes (including ETbR mRNA expressions). Even though not being essential herein the explanation of the mechanism of action of the aforementioned complexes, different way of action could be supposed: a binding between H atoms of TNF alpha may be formed as the molecular complex tridimensionally resembles TNFα tridimensional configuration, or the complexes may bind TNF Alpha receptors from keratinocytes and other cutaneous cells (FIGS. 3, 4). The formulations comprising the complexes according to the invention have both intracellular and extracellular actions. The complex will translocate into the cell by pinocytosis or endocytosis processes since formulations according to the invention have a pH comprised between 3.0 and 5.5 and more precisely between 3.7 and 4.4; this pH difference between intra and extracellular environment allows substance crossing from extracellular to intracellular environment by pinocytosis or endocytosis. The new complexes should have a direct action on TNFα production stimuli. Said complexes being made of natural compounds would allow inhibition of melanine overproduction without cytotoxic effects through tyrosinase inhibition, furthermore, would destroy TNFα production selectively, or its side effects at cutaneous level and/or in the exacerbation of diseases involving TNFα, without affecting the normal organism immune response, while would not act on the expression of the gene inducing tyrosinase enzyme production. New complexes allow skin to show a uniform color by re-equilibrating and making melanine distribution uniform at epidermis level, and further act in the repair of cellular DNA damage following exposure to actinic radiations. In view of the fact that melanine synthesis may be stimulated by TNFα, the action of the complexes object of the present invention would selectively regulate the expression at the different above said levels without changing immune response. The possible mechanism of action of the complexes according to the present invention may be confirmed by observing their similar suramin structure. In fact it has been confirmed that suramin has an inhibitory effect on TNF alpha receptors (Biochem Pharmacol 1999 Sep. 1; 58 (5): 851-9, “Inhibition of tumor necrosis factor alpha/alpha receptor binding by structural analogues of suramin” di Mancini F et al.). By observing suramin molecule, it recalls the structure of the complexes according to the present invention because of the four rings, so that, while the fourth ring of suramin is benzenic, in the molecule according to the present invention it is represented by the lactonic ring of vitamin C, furthermore the two molecules present a certain tridimensional similitude even if suramin being constituted by a same molecule in both dextrorotatory and levorotatory forms is bigger (see FIG. 2). Probably inside the cell, the molecular complex object of the present invention exerts a bleaching action by a direct action on tyrosinase, thus reducing TNFα effects and repairing mitochondrial DNA damages. Whilst suramin action is more anti-inflammatory than directed to DNA repair. Furthermore, suramin is used as antiblastic and in chemotherapy, therefore its side effects are relevant (Cancer. 1999 Nov. 1; 86(9):1733-41. Combining suramin and a chimeric toxin directed to basic fibroblast growth factor receptors increases therapeutic efficacy against human melanoma in an animal model. Davol P A, Garza S, Frackelton A R Jr.) (Anat Embryol (Berl). 2003 February; 206 Teratogenic effects of suramin on the chick embryo. Manner J, Seidl W, Heinicke F, Hesse H).

The complex according to the present invention has shown an action never seen before, in fact it promotes collagen synthesis, repairs cellular damages, stabilize and strengthen the action of all substances used in medical, dermatologic and cosmetic field, allowing the penetration of the substances complexed, enhancing their positive features, strengthening therapeutic and cosmetic effectiveness. The molecular complex according to the present invention would have an intracytoplasmic action (also acting on protein biosynthesis and, simultaneously, has an action inside cell mitochondria and nucleus stabilizing DNA and enhancing DNA resistance to nociceptive and, whether DNA would be already damaged, it will encourage a quick repair or apoptosis.

It is therefore an object of the present invention a process for the preparation of a molecular complex comprising ascorbic acid or its derivative, oleuropeina or its derivative and arbutine or its derivative comprising the following steps:

a) solubilizing into water a suitable amount of ascorbic acid or its derivative, at a temperature between 40 and 50° C.; b) adding an equal amount of oleuropeina or its derivative to ascorbic acid or its derivative and then alpha or beta arbutine or its derivative by mixing until a clear solution is obtained. The molecular complex may further comprise a ionene polymer, therefore the process for the preparation of the complex may comprise a further step c) wherein ionene polymer in a variable concentration ranging from 0.01 to 20% is added. The solution may be prepared with a stirrer heater and by an homogenizer up to 4.500 turns, in fact this aids full homogenization of the product and tri- or tetramolecular complex formation. During reaction between complex components water formation may be observed. Oleuropeina may be added using an olea europea extract, preferably, an aqueous extract. Ascorbic acid may be L-ascorbic acid whilst ascorbic acid derivatives may be esters of ascorbic acid with fatty acids, such as, ascorbyl palmitate and their salts. Ionene polymers may have general formula (III):

[—N(CH₃)₂—(CH₂)ₓ—N(CH₃)₂—(CH₂)_{y}—].2Z⁻ (III)

wherein x and y are integer number comprised between 1 and 10, preferably between 2 and 5, while Z is an halide, as for example, Br or Cl. Ionene polymer may be obtained by reaction of 1,4-dichlorobutane with tetramethylendiamine. Furthermore, ionene polymers may be selected among those obtained by reaction of 1,4-dichlorobutane with poly(oxyethylene(dimethylimino)-ethylene(dimethylimino)ethylene dihalides), poly(2-hydroxyethylene-dimethylimino-2-hydroxypropylene-dimethylimino methylene)dihalides, poly[{alkyl-(3-ammoniopropyl)imino}trimethylene dihalides], poly-[dialkyl-imino)ethylene halides] or with poly-[(hydroxy-dialkyl-imino)ethylene halides.

Weight percentages of molecular complex forming components ascorbic acid or its derivatives, oleuropeina or its derivatives and arbutine or its derivatives, ionene polymer, range each independently from 0.01 to 35%, preferably from 0.5% to 20%, more preferably from 1% to 12%.

It is an object of the present invention a molecular complex comprising ascorbic acid, such as L-ascorbic acid, or its derivative, oleuropeina or its derivative, alpha or beta, arbutine or its derivative obtainable by the above defined process. The ascorbic acid derivative may be an ester of ascorbic acid with fatty acids, such as ascorbyl palmitate or its salts.

The molecular complex may further comprise ionene polymer. The latter may have general formula (III):

[—N(CH₃)₂—(CH₂)ₓ—N(CH₃)₂—(CH₂)_{y}—].2Z⁻ (III)

wherein x and y are integer number comprised between 1 and 10, preferably between 2 and 5, while Z is an halide, for example Br or Cl. Particularly, ionene polymer may be obtained by reaction of 1,4-dichlorobutane with tetramethylendiamine. Examples of ionene polymers are those obtained by reaction of 1,4-dichlorobutane with poly(oxyethylene(dimethylimino)-ethylene(dimethylimino)ethylene dihalides), poly(2-hydroxyethylene-dimethylimino-2-hydroxypropylene-dimethylimino methylene)dihalides, poly[{alkyl-(3-ammoniopropyl)imino}trimethylene dihalides], poly-[dialkyl-imino)ethylene halides] or with poly-[(hydroxy-dialkyl-imino)ethylene halides. Ionene polymer is an important active principle for its properties in medical field, is herein used as antiaging agent as, in combination with the other compounds, it forms new complexes never used in medical, pharmaceutical and cosmetic fields.

Weight percentages of the molecular complex forming components ascorbic acid or its derivative, oleuropeina or its derivative and arbutine or its derivative, ionene polymer range between 0.01 and 35%, preferably between 0.5% and 20%, more preferably between 1% and 12%, while ionene polymer ranges between 0.01% and 20%.

Oleuropeina of the complex may be provided using an olea europea extract, preferably an aqueous extract.

The molecular complex according to the invention may be advantageously used in medical field.

It is a further object of the present invention a composition comprising a molecular complex as above defined as active principle along with one or more adjuvants and/or excipients pharmaceutically or cosmetically acceptable. The composition may further comprise hormones, such as melatonin.

The composition according to the invention may further comprise tyrosol and hydroxytyrosol. Furthermore, the composition may comprise vitamin A and/or vitamin E (alpha tocopherol), preferably in its acetate form, for example, at a concentration between 0.01% and 20%, preferably between 2% and 7%.

The composition according to the invention may further comprise Aloe and/or Opunzia and derivatives therefrom. Furthermore, the composition may comprise bleaching substances such as, for example, cogic acid, azelaic acid, liquorice extracts or may contain triphosphate adenosine or nicotinamide as substances providing cellular energy readily usable (ADP or ATP or NADH). The composition may further comprise one or more compounds selected from the group consisting of glycolic, lactic, fumaric, tartaric, L-aspartic, gluconic, fitic, trans and cis-retinoic acids, for example, at a concentration between 0.5% and 20%, while trans and cis-retinoic acids at a concentration between 0.01% and 5%. Particularly, fitic acid make the cream red.

In addition, the composition according to the present invention may comprise one or more emollients, flowings, emulsifiers, preservatives, surface active agents, fungicides. For example, emollients are cetyl esters at a concentration between 0.1% and 10%, preferably from 5% to 9%, flowings are selected from stearyl alcohol at a concentration between 0.1% and 15%, preferably from 5% to 12%, or cheryl alcohol at a concentration between 5% and 12%, emulsifiers are selected from cetyl alcohol at a concentration between 0.1% and 6% preferably from 2% to 5%, glycerine at a concentration between 0.1% and 20%, preferably from 2% to 20%, sodium lauryl sulphate from 0.1% to 1.5%, preferably from 0.5% to 1.0%.

Cetyl esters are of synthetic origin and in any case not distinguishable from waxes derived from natural spermaceti as far as chemical composition and properties are concerned. Said esters consist of a mixture of esters of fatty acids containing between 14 and 18 atoms of carbon along with alcohols and they can be included in the formulations as emollients or “softening agents”. Cetyl ester, stearyl alcohol, cetyl alcohol, and glycerine form a moisturizing basic cream promoting the application on the skin with positive effects. The following three components have shown to improve stability of the formulations according to the invention.

Deionised or distilled water may be present in the formulations according to the present invention as inert carrier acting as diluent and at the same time having wetting properties.

Preservative to be used may be methyl paraben at a concentration between 0.1% and 0.4%, preferably between 0.05% and 0.3%. Surface active agents may be present at a concentration between 0.01% and 0.15%, preferably between 0.05% and 0.12%. Fungicide may be propyl paraben at a concentration between 0.01% and 0.1%, preferably between 0.02% and 0.05%.

The composition according to the invention may be prepared in the form of serum, solution, gel, emulsions, creams, spray, tablets, capsule, suppository, medical devices, such as gauzes, bandages, plasters, silicon bars, vials for intravenous, intramuscular, ipo- or intradermic infiltration.

According to a preferred embodiment of the present invention, the composition comprises between 0.01% and 20% in weight of each component ionene polymer, Olea Europea, Arbutine, L-ascorbic acid; between 0.5% and 20% in weight of an acid selected from the group consisting of cogic, azelaic, glycolic, lactic, fumaric, tartaric, L-aspartic acid, gluconic acids; between 0.01% and 5% in weight of trans-cis retinoic acid (Vitamin A); between 0.5% and 10% in weight of Vitamin E acetate; between 0.1% and 10% in weight of any naturally occurring or synthetic vitamin; along with one or more adjuvants and/or excipients pharmaceutically or cosmetically acceptable. Another preferred embodiment of the present invention consists in 2.1% ionene polymer, 5% Alpha Arbutine, 5% Ascorbic Acid (LAA), 5% Olea Europea, 0.08% cis-retinoic acid (Vitamin A), 0.5% Vitamin E acetate, 4.2% cogic Acid, 8.4% Cetyl ester, 4% Cetyl alcohol, 10% Glycerine, 0.2% Methyl Paraben, 0.02% Propyl Paraben, 0.1% cationic surface-active Quaternium/15, 2.5% laurylsulfate sodium, Deionised water up to 100%. Another preferred embodiment of the present invention consists in a composition of a bleaching cream that may be useful to repair DNA damages or in pre-cancerosis, consisting of a trimolecular complex with 12% Alpha Arbutine, L ascorbic acid and Olea Europea, comprising 34-50% Water, 0.2-4% Xantan gum, 0.4-1% EDTA, 5-20% Glycerine, 2-10% Transcutol, 1.0% Amigdalol, 3-8% Macadamia, 3-10% Isopropyl miristate, 1-6% Cetylic 3M, 3-10% Fattilan, 0.05-2% BHT, 0.4-2% UBF, 0.1-3% Dimeticon, 0.05-1% Opuntia, 12% Arbutine, 12% Olea, 12% L-ascorbic acid, between 1.0% and 20.0% Sodium hydrate, 0.4-4% Grapefruit seeds.

The compositions as above defined may be advantageously used in medical, pharmaceutical and cosmetic field.

Another aspect of the present invention relates to the use of the molecular complex as above defined and compositions as above defined for cosmetic or medical treatment, depending on the concentrations being employed, for example, of the skin and of unaesthetisms, such as against skin aging, wrinkles, melasma, for the treatment of cutaneous hyperpigmentation and precancerosis including actinic keratosis.

Further object of the present invention is the use of molecular complex and of the compositions according to the invention for the preparation of a medicament for the topical treatment of skin diseases, mucous, and serosa also together with collagen from any plant or animal source or fibrin glue, for the anti-inflammatory treatment, for the treatment of actinic keratosis, for the treatment of wound healing phase and for the prevention of post-inflammatory hyperpigmentations, for the treatment of sores, and possibly in association with Melatonin, for the treatment of diabetic cutaneous ulcers, and of acute or chronic lesions of skin, for the treatment of lesions of oral mucous, for prevention and treatment of skin tumours, for the treatment of psoriasis, for the treatment of folliculitis.

The present invention will be now described, for illustrative but not limitative purposes, according to its preferred embodiments, with particular reference to the figures of the enclosed drawings and examples, wherein:

FIG. 1 shows the interactions between the molecules of the tetramolecular complex ascorbic acid, arbutine, oleuropeina and ionene polymer.

FIG. 2 shows the formula of the tetramolecular complex according to the invention and the comparison with suramin.

FIG. 3 shows the tridimensional structure of the trimolecular complex.

FIG. 4 shows the binding between TNF alpha receptor and trimolecular complex.

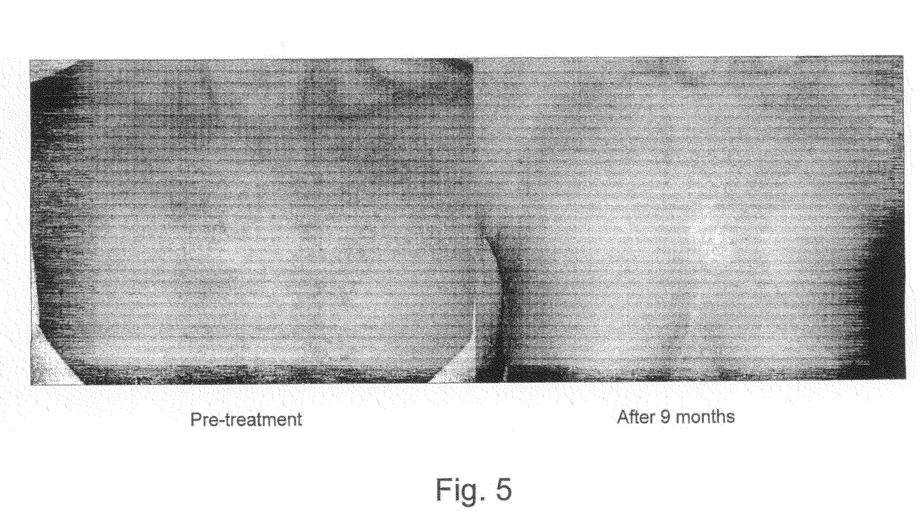

FIG. 5 shows the results of 9 months treatment of cutaneous hyperpigmentation and actinic keratosis with a composition according to the invention.

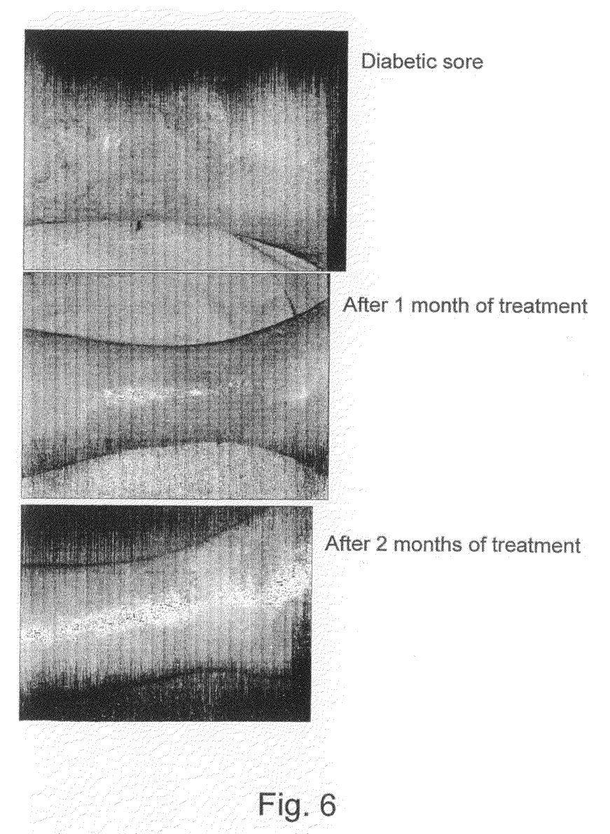

FIG. 6 shows the recovery process of a diabetic sore by using a composition according to the invention.

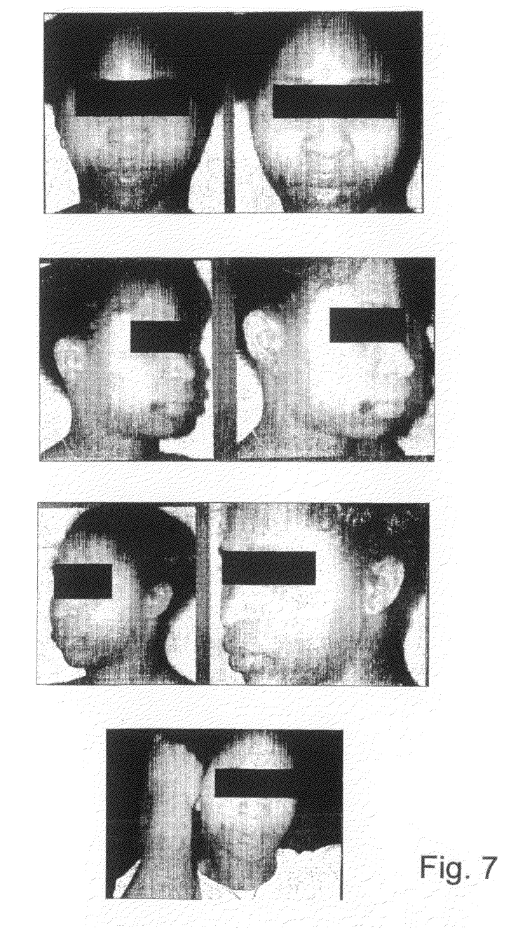

FIG. 7 shows the bleaching action on the skin by using a composition according to the invention after 6 weeks treatment.

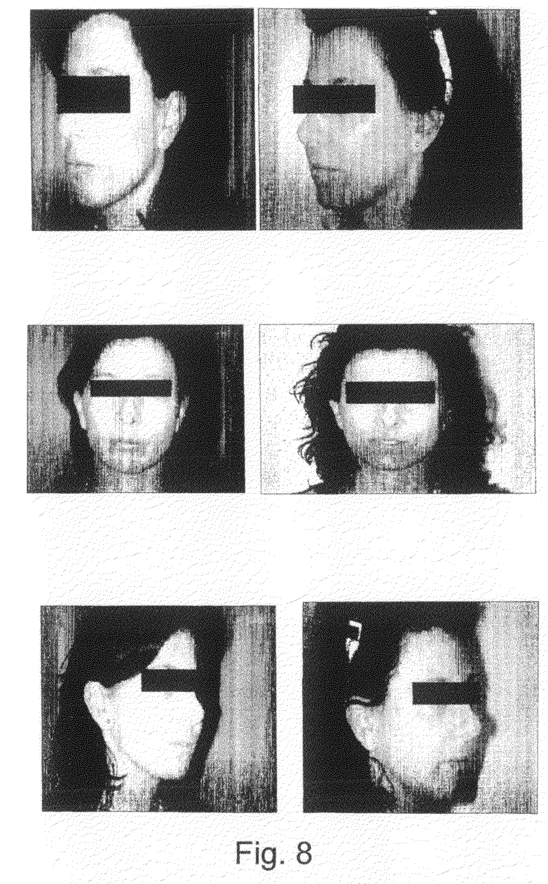

FIG. 8 shows the results of the treatment of a face burn with a composition according to the invention.

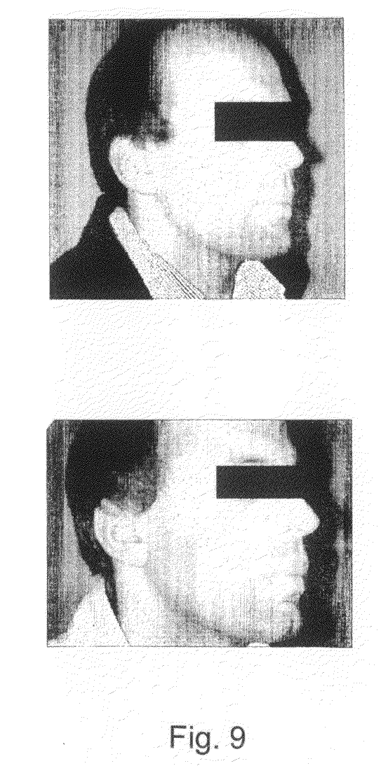

FIG. 9 shows the results of the treatment of face folliculitis with a composition according to the invention.

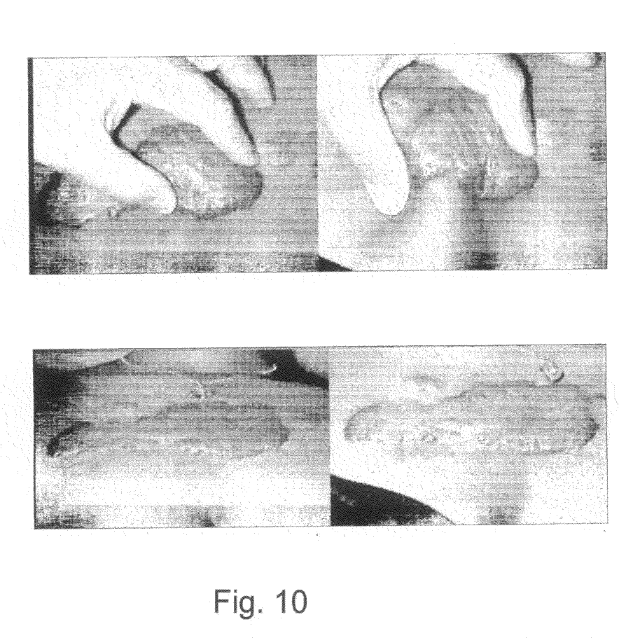

FIG. 10 shows the results of the treatment of thorax cheloids and bleaching with a composition according to the invention.

### EXAMPLE 1

### Composition According to the Invention

Bleaching cream containing a trimolecular complex with 12% Alpha Arbutine, L ascorbic acid and Olea Europea incorporating:

### 34.0% Water,

0.2% Xantan gum,

### 0.04% Edta,

### 5.0% Glycerine,

### 5% Transcutol,

### 1.0% Amigdalol,

### 3.0% Macadamia,

3.0% Isopropyl myristate,

### 3.0% Cetylic 3M,

### 6.41% Fattilan,

### 0.5% BHT,

### 0.4% UBF,

### 1.0% Dimethycone,

### 0.05% Opuntia,

### 12.0% Arbutine,

### 12.0% Olea,

12.0% L-Ascorbic acid,
1.0% Sodium hydrate,
0.4% Grapefruit seeds.

The preparation of the composition comprises the following steps:

1) FIRST PHASE: Arbutine, Olea and Vitamin C were dissolved in water at a 40/50° C. temperature, homogenized and subsequently solubilized until the solution is clarified.
2) SECOND PHASE: Xantan gum, glycerine, transcutol, tetrasodic etda were solubilized in water at a 70° C. temperature.
3) THIRD PHASE: Amigdalol, macadamia, isopropylmyristate, cetylic 3M, fattilan, Dimethycone, BHT were solubilized a part up to a temperature of 80° C.
4) FOURTH PHASE: The second phase, third phase were mixed by homogenization at 3.500 turns and the whole homogenized.
5) FIFTH PHASE: Nr. 1 phase, that is tetramolecular complex, Vit. E, was added to the composition when temperature reaches 50° C., then pH was measured, and optionally sodium hydrate, and grapefruit seeds were added. Thus, a cream composition in high absorbing form was obtained.

### EXAMPLE 2

### Treatment of Cutaneous Hyperpigmentation by Using Composition According to Example 1 (12% Alpha Arbutine Containing Cream)

53 years-old woman N. B, with diffuse precancerosis and possible pigmentous xeroderma. The composition was administered daily, twice a day, and patient was educated to spread the cream uniformly and to double the application on spots or precancerosis, topical treatment was carried out in association with two vascular Laser passage one three months later the other for the treatment of cutaneous teleangectasys in peri lesion sites. After 9 months from the beginning of the treatment results show the powerful bleaching action and the main effect on cutaneous precancerosis. The scar of a previous surgical operation nearly disappeared due to the collagen stimulating action of the product object of the invention. It is even more the action on precancerosis and actinic keratosis that will disappear after this alone topical treatment.

FIG. 5 shows how after 9 months of treatment a uniform cutaneous colour has been obtained, without paradoxical hyperpigmentation or activation of inflammatory processes due to overdosage.

### EXAMPLE 3

### Treatment of a Diabetic Sore by Using the Composition of Example 1

65 years old patient affected by a diabetic sore treated for over 9 months with other products in several centre. Treatment starts with a topical application of the Cream object of the present patent application, once a day, in association with melatonin, and Vit. C serum and surgical curettage of sores.

FIG. 6 shows the recovery process of a diabetic sore after two months of treatment.

### EXAMPLE 4

### Cheloid Treatment and Skin Bleaching Using the Composition of Example 1

35 years old patient of black race and thorax cheloids caused by a previous accidental burn occurred in pediatric age; these cheloids were presented as solid neoformation, anelastic, on palpation feels tough, not movable, rather, fixed on subcutaneous plane. The patient was treated daily at a dosage of 1 g with the new cream object of the present patent application to be applied twice a day. The patient doubles the application on darker skin regions.

Cream was applied also on thorax cheloids, twice a day.

After 7 weeks, not only skin bleaching was noted, but also even more cheloid became softer. This is noted mainly by the touch and finger stapling manoeuvre (FIGS. 7 and 10)

Cream was spread uniformly on the whole face, and doubled on spots. It was also noted the whole bleaching of the thorax region.

### EXAMPLE 5

### Treatment of Face Burn Using the Composition of Example 1

39 years old woman patient affected by melasma, after extensive sun esposition shows a second grade surface burn. The patient was treated with topical applications of the cream object of the present patent application in association with melatonin. Neither anti-inflammatory or steroids or other kind of re-epithelizing agents were employed as the product is already active per se. Patient has not developed after seven days of treatment any kind of post-inflammatory hyperpigmentation, rather, face extended melasma resulted noticeably reduced. The application was carried out as follows: Cream containing tetramolecular complex object of the present invention, once in the morning, doubling on the spots. The patient carried out topical applications of the cream containing melatonin in the evening. FIG. 8 shows bleaching and re-epithelization after 4 weeks. It needs to be underlined that no steroid topical treatment was carried out to show the anti-inflammatory action of Melatonin associated to this trimolecular complex.

Notably, the new molecule spread on face twice a day not only has treated melasma and the burn, but it avoids also post inflammatory hyperpigmentation outcome, that would be sure in a subject affected by melasma. Furthermore, post inflammatory hyperpigmentation was partly caused by TNF Alpha, this is another evidence of beneficial effects of the new molecule.

### EXAMPLE 6

### Treatment of Face Folliculitis Using the Composition of Example 1

35 years old patient, affected by folliculitis face diffused near beard region. Furthermore, diffused hyperpigmentation near beard, forehead and periocular regions was observed. The patient has carried out treatment once a day by spreading the cream uniformly containing the complex object of the present patent application once a day in an amount of ½ g doubling application on spots and folliculitis. It is noted that after four weeks folliculitis were dissolved, and the skin acquires a uniform colour, with an improved vascularization and compactness caused by collagen stimulation (FIG. 9).

1. Process for the preparation of a molecular complex comprising ascorbic acid or its derivative, oleuropeina or its derivative and arbutine or its derivative comprising the following steps:
a) solubilizing into water a suitable amount of ascorbic acid or its derivative, at a temperature between 40 and 50° C.;
b) adding an equal amount of oleuropeina or its derivative to ascorbic acid or its derivative and subsequently arbutine or its derivative by mixing until a clear solution is obtained.

2. Process according to claim 1, for the preparation of a molecular complex further comprising an ionene polymer, wherein the step of adding an equal amount of oleuropeina or its derivative comprises adding an aqueous olea europea extract, and the process comprising a further step c) of adding an ionene polymer with a concentration ranging from 0.01% to 20%.

3-4. (canceled)

5. Process according to claim 1, wherein the ascorbic acid is L-ascorbic acid.

6. Process according to claim 1, wherein the ascorbic acid derivatives are ascorbyl palmitate and their salts.

7. (canceled)

8. Process according to claim 1, wherein the arbutine is alpha or beta arbutine.

9. Process according to claim 2, wherein the ionene polymers have a general formula:
[—N(CH3)2-(CH2)[chi]-N(CH3)2-(CH2)y-]2Z-
wherein x and y are integer numbers between 1 and 10, and Z is Br or Cl.

10. (canceled)

11. Process according to claim 2, wherein ionene polymer is obtained by reaction of 1,4-dichlorobutane with tetramethylendiamine.

12. Process according to claim 2, wherein ionene polymers are selected among those obtained by reaction of 1,4-dichlorobutane with poly(oxyethylene(dimethylimino)-ethylene(dimethylimino)ethylene dihalides), poly(2-hydroxyethylene-dimethylimino-2-hydroxypropylene-dimethylimino methylene)dihalides, poly[{alkyl-(3-ammoniopropyl)imino}trimethylene dihalides], poly-[dialkyl-imino)ethylene halides] or with poly-[(hydroxy-dialkyl-imino)ethylene halides.

13. Process according to claim 1, wherein weight percentages of the molecular complex forming components ascorbic acid or its derivative, oleuropeina or its derivative and arbutine or its derivative range between 1 and 12%.

14-15. (canceled)

16. Molecular complex comprising ascorbic acid or its derivative, oleuropeina or its derivative, alpha or beta, arbutine or its derivative obtainable by the process as defined according to claim 1.

17. Molecular complex according to claim 16, wherein the ascorbic acid is L-ascorbic acid.

18. Molecular complex according to claim 16, wherein the ascorbic acid derivative is ascorbyl palmitate or its salt.

19. (canceled)

20. Molecular complex according to claim 16, wherein arbutine is alpha or beta arbutine.

21. (canceled)

22. Molecular complex according to claim 16, further comprising ionene polymer having a general formula:
[—N(CH3)2-(CH2)x-N(CH3)2-(CH2)y-]-2Z-
wherein x and y are integer number between 1 and 10, and Z is Br or Cl.

23. (canceled)

24. Molecular complex according to claim 22, wherein the ionene polymer is obtained by reaction of 1,4-dichlorobutane with tetramethylendiamine.

25. Molecular complex according to claim 22, wherein the ionene polymer is selected among those obtained by reaction of 1,4-dichlorobutane with poly(oxyethylene(dimethylimino)-ethylene(dimethylimino)ethylene dihalides), poly(2-hydroxyethylene-dimethylimino-2-hydroxypropylene-dimethylimino methylene)dihalides, poly[{alkyl-(3-ammoniopropyl)imino}trimethylene dihalides], poly-[dialkyl-imino)ethylene halides] or with poly-[(hydroxy-dialkyl-imino)ethylene halides.

26. Molecular complex according to claim 16, wherein weight percentages of the components ascorbic acid or its derivative, oleuropeina or its derivative and arbutine or its derivative forming the molecular complex range between 0.01 and 35%.

27. Molecular complex according to claim 16, wherein weight percentages of said components range each independently between 0.5% and 20%.

28. Molecular complex according to claim 16, wherein weight percentages of said components range each independently between 1% and 12%.

29. Molecular complex according to claim 22, wherein ionene polymer weight percentage ranges between 0.01% and 15%.

30. Molecular complex according to claim 16, wherein oleuropeina is from olea europea aqueous extract.

31-32. (canceled)

33. Composition comprising the molecular complex as defined in claim 16 as an active principle along with one or more adjuvants and/or excipients pharmaceutically active or cosmetically acceptable and further comprising hormones.

34. (canceled)

35. Composition according to claim 33, wherein the hormone is melatonin.

36. Composition according to claim 33, further comprising tyrosol and hydroxytyrosol.

37. Composition according to claim 33 further comprising vitamin A and/or vitamin E present at a concentration between 0.01% and 20%.

38. (canceled)

39. Composition according to claim 37, wherein vitamin A and/or vitamin E are present at a concentration between 2% and 7%.

40. Composition according to 33, further comprising Aloe and/or Opunzia.

41. Composition according to 33, further comprising bleaching substances selected from the group consisting of cogic acid, azelaic acid, liquorice extracts.

42. (canceled)

43. Composition according to claim 33, further comprising ADP, ATP or NADH.

44. Composition according to claim 33, further comprising one or more compounds selected from the group consisting of glycolic, lactic, fumaric, tartaric, L-aspartic, gluconic, fitic, trans and cis-retinoic acids, said one or more components being present at a concentration between 0.5% and 20%, while retinoic acid is present at a concentration between 0.01% and 5%.

46. Composition according to 33, further comprising one or more emollients, flowings, emulsifiers, preservatives, surface active agents, fungicides.

47. Composition according to claim 46, wherein emollients are cetyl esters at a concentration between 5% and 9%.

48. (canceled)

49. Compositions according to claim 46, wherein flowings are selected from stearyl alcohol at a concentration between 5% and 12%, and Cheryl alcohol at a concentration between 5% and 12%.

50. (canceled)

51. Compositions according to claim 46, wherein emulsifiers are selected from cetyl alcohol at a concentration between 2% and 5%, glycerine between 2% and 20%, sodium lauryl sulphate from 0.5% to 1.0%.

52-54. (canceled)

55. Compositions according to claim 46, wherein the preservative is methyl paraben at a concentration between 0.05% and 0.3%.

56. (canceled)

57. Compositions according to claim 46, wherein surface active agents are present at a concentration between 0.05% and 0.12%.

58. (canceled)

59. Compositions according to claim 46, wherein fungicide is propyl paraben at a concentration between 0.02% and 0.05%.

60. (canceled)

61. Composition according to claim 33 in the form of serum, solution, spray, gel, emulsions, creams, tablets, capsule, suppository, medical devices in the form of gauzes, bandages, plasters, silicon bars, vials for intravenous, intramuscular, ipo- or intradermic infiltration.

62. (canceled)

63. Composition according to claim 33 comprising between 0.01% and 20% in weight of each component Ionene polymer, Olea Europea, Arbutine, L-ascorbic acid; between 1.5% and 10% in weight of an acid selected from the group consisting of cogic, azelaic, glycolic, lactic, fumaric, tartaric, L-aspartic, gluconic acids; between 0.01% and 5% in weight of trans-cis retinoic acid (Vitamin A); between 0.5% and 10% in weight of Vitamin E acetate; between 0.1% and 10% in weight of any naturally occurring or synthetic vitamin; along with one or more adjuvants and/or excipients pharmaceutically or cosmetically acceptable.

64. Composition according to claim 33 consisting of 2.1% ionene polymer, 5% Alpha Arbutine, 5% Ascorbic Acid (LAA), 5% Olea Europea, 0.08% cis-retinoic acid (Vitamin A), 0.5% Vitamin E acetate, 4.2% cogic Acid, 8.4% Cetyl ester, 4% Cetyl alcohol, 10% Glycerine, 0.2% Methyl Paraben, 0.02% Propyl Paraben, 0.1% cationic surface-active Quaternium/15, 2.5% laurylsulfate sodium, Deionised water up to 100%.

65. Composition according to claim 33 consisting of 34-50% Water, 0.2-4% Xantan gum, 0.4-1% EDTA, 5-20% Glycerine, 2-10% Transcutol, 1.0% Amigdalol, 3-8% Macadamia, 3-10% Isopropyl miristate, 1-6% Cetylic 3M, 3-10% Fattilan, 0.05-2% BHT, 0.4-2% UBF, 0.1-3% Dimeticon, 0.05-1% Opuntia, 12% Arbutine, 12% Olea, 12% L-ascorbic acid, between 1.0% and 20.0% Sodium hydrate, 0.4-4% Grapefruit seeds.

66. Composition as defined in for use in medical field.

67-81. (canceled)
